(19) United States
(12) Patent Application Publication
(10) Pub. No.: US 2012/0059262 A1
(43) Pub. Date: Mar. 8, 2012

(54) SPECTRAL DOPPLER ULTRASOUND IMAGING DEVICE AND METHOD FOR CONTROLLING SAME

(75) Inventor: David Clark, Derry, NH (US)

(73) Assignee: KONINKLIJKE PHILIPS ELECTRONICS N.V., Eindhoven (NL)

(21) Appl. No.: 13/266,784

(22) PCT Filed: Apr. 22, 2010
(86) PCT No.: PCT/IB10/51773

§ 371 Date: Oct. 28, 2011

(30) Foreign Application Priority Data
Apr. 28, 2009 (US) 61173303

(51) Int. Cl. A61B 8/00

(52) U.S. Cl. 600/440

(57) ABSTRACT

A method for controlling a spectral Doppler ultrasound imaging device adapted for operating both in a spectrum-live Doppler measurement mode (S3) and in an image-live measurement mode (S1, S6) is proposed. In accordance with the present invention, the method comprises automatically switching to the image-live measurement mode (S6) upon detection (S5) of a change of a sampled region. A change of a sampled region may be indicated by an operator by actuating a sampled region control device such as a trackball, angle knob or sample gate controller and, upon such actuation, the spectral Doppler ultrasound imaging device may automatically switch to the image-live measurement mode (S6) to allow for adapting or correcting of a position and/or orientation of the sampled region. After the sampled region has been positionally stable for a predetermined duration, the spectral Doppler ultrasound imaging device may be switched back (S7) to the spectrum-live measurement mode (S3). Thereby, unnecessary manual toggling between different measurement modes may be avoided and quantitative spectrum-live Doppler measurement (S3) is enabled being interrupted only when necessary, i.e. when a sampled region is changed.

### FIELD OF THE INVENTION

The invention relates to a method for controlling a spectral Doppler ultrasound imaging device adapted for operating both in a spectrum-live Doppler measurement mode and in an image-live measurement mode. Furthermore, the invention relates to such spectral Doppler ultrasound imaging device being adapted for performing the inventive method, to a computer program product for accordingly controlling a spectral Doppler ultrasound imaging device when executed on a computer and a computer-readable medium having such computer program product stored thereon.

### BACKGROUND OF THE INVENTION

Ultrasound imaging devices have been developed for qualitatively and quantitatively imaging both static structures within a sampled region and dynamic motions within the sampled region. Therein, ultrasound is emitted towards the sampled region and echoes reflected within the sampled region are detected. The sampled region, sometimes also referred to as region of interest, may be a portion of a body of a patient. A dynamic motion within the sampled region may be e.g. the flow of blood in vessels within the patient's body.

In a first measurement mode, the ultrasound imaging device may be adapted for acquiring a grey-scale or color 2D image of the sampled region. Such 2D image is preferably acquired in real-time. This measurement mode is herein also referred to as “image-live measurement mode”.

In a second measurement mode, the ultrasound imaging device is adapted for quantitative analysis of motions within the sampled region. Such motions frequently result from blood flow within vessels and may be used to indicate characteristics of vascular or cardiac structures. Therein, ultrasound is emitted towards the sampled region and Doppler effects resulting from the relative motion within the sampled region with respect to the static imaging device are detected. From such spectral Doppler measurements, quantitative analysis of velocities of motions within the sampled region may be derived. Such measurement mode is herein also referred to as “spectrum-live Doppler measurement mode”.

In both, the image-live and the spectrum-live measurement mode, a corresponding part of a displayed feature (image or spectrum) is continually updated at a rapid rate of e.g. more than 10 Hertz.

The position and angle of the ultrasound waves emitted towards the sampled region for spectral and audio Doppler ultrasound is in most cases manually adjusted based on the grey or color 2D image. For this purpose, deliberately moving or attempting to hold an ultrasound transducer's position, patient respiration and/or measuring multiple vessels or locations in an extended blood vessel or cardiac scan generally requires repeatedly using an ENTER or UPDATE key to manually toggle between the spectrum-live mode and the image-live mode to adjust the position of the sampled region for example with a trackball or an angle knob provided on the ultrasound imaging device.

The toggling between the spectrum-live mode and the image-live mode may complicate a user's workflow during the Doppler part of an ultrasound exam due to the conventionally necessary repetitive use of the ENTER/UPDATE key to manually toggle between the spectrum-live and image-live mode for adjusting the position or angle of the sampled region.

In a conventional approach to overcome such frequent toggling between spectrum-live and image-live mode, a single frame image update may be initiated by a software timer, ECG trigger or user control adjustment. However, this does not show the flow dynamics, is different or unlikely to catch a jet or systolic pulse, may interrupt the several seconds needed for Doppler spectrum measurements and may waste most of an update time with slewing and settling a transmit voltage. Thus, many users may prefer to manually toggle between the spectrum-live and image-live measurement mode.

In another approach, line interleaved (triplex) and frame interleaved (gap fill) techniques can be used to display image-live and spectrum-live measurement results simultaneously. However, these techniques inevitably involve compromises in velocity scale, frame rate, sensitivity, artefacts and/or measurement accuracy. The clinical applications that use Doppler ultrasound imaging most intensively and quantitatively such as vascular and cardiac applications are also the applications that are the least tolerant to compromises concerning the spectral Doppler ultrasound imaging.

In a further approach, automatic sample region detection techniques have been developed. However, since such techniques depend on acquiring image-live measurements they have similar issues of switching between spectrum-live and image-live mode as described further above and may compromise both.

### SUMMARY OF THE INVENTION

It is an object of the invention to provide a method for controlling a spectral Doppler ultrasound imaging device adapted for operating both in a spectrum-live Doppler measurement mode and in an image-live measurement mode wherein the method may simplify the use of the spectral Doppler ultrasound imaging device. It is another object of the invention to provide a spectral Doppler ultrasound imaging device adapted for operating in accordance to such simplifying method, a computer program product adapted to control, when executed on a computer, a spectral Doppler ultrasound imaging device in accordance with such simplifying method and a computer-readable medium having such computer program product stored thereon.

The invention is defined by the independent claims. Advantageous embodiments are defined in the dependent claims.

According to a first aspect of the present invention, a method for controlling a spectral Doppler ultrasound imaging device adapted for operating both in a spectrum-live Doppler measurement mode and in an image-live measuring mode is proposed. The method comprises automatically switching to the image-live measurement mode upon detection of a change of a sampled region.

According to a second aspect of the present invention, a spectral Doppler ultrasound imaging device being adapted to perform the method according to the first aspect of the invention is proposed.

According to a third aspect of the present invention, a computer program product comprising a program code which, when executed on a computer, is adapted to control a spectral Doppler ultrasound imaging device according to the method of the first aspect of the invention is proposed.

According to a fourth aspect of the present invention, a computer-readable medium having a computer program product according to the third aspect of the present invention is proposed.

The present invention may be seen as based on the following idea:

Embodiments of the invention deal with spectral Doppler ultrasound imaging. Such spectral Doppler ultrasound imaging may allow accurate quantitative measurement of e.g. blood flow velocities, compared to other kinds of imaging modalities.

To do such quantitative measurements, a sampled region, herein also sometimes referred to as gate or sample volume or sample gate, may have to be positioned accurately in a region of interest within a medical diagnostic ultrasound live-image. An alternative mode is continuous-wave (CW) Doppler, where the sampled region is an entire straight line from the transducer face, rather than a sample gate as with pulsed-wave (PW) Doppler. Both Doppler modes are applicable for the present invention.

Some conventional spectral Doppler ultrasound imaging systems rely on the operator to perform the task of positioning, some other systems propose a semi or full automation of this task. In any case, a general aim of these systems may be to show in real-time the spectrum-live Doppler ultrasound image and the image-live color or grey-scale ultrasound image. However, this may be very difficult because both measurement modes may need different types of ultrasound firing. Line-interleave and frame interleave techniques can be discussed together as interleaving one or more image lines between one or more Doppler lines, at rapid enough rate and with interpolation to continually and rapidly update both image and spectrum display. Line interleave, i.e. alternately firing a line of spectral a color Doppler , and frame interleave may not be provided in real-time and compromises may have to be done. However, such compromises necessary for realizing semi or fully automatic Doppler ultrasound imaging may be unacceptable for certain medical applications.

In the case, the ultrasound imaging system relies on the help of an operator, a sample gate position and/or angle may be manually adjusted based on a grey and/or color 2D live-image. However, generally, because the patient is at least slightly moving or due to patient's respiration movements, unintentional drift of the ultrasound transducer position or angle or in case of a need to measure motion of blood flow in multiple locations in a patient's body, the operator may have to repeatedly use an ENTER or UPDATE key to manually toggle between spectrum-live and image-live and thus adjust the sample gate for example with a trackball or an angle knob. Although an accurate measurement may be performed in this manual way, the procedure of toggling between the two imaging modes may be time-consuming and tedious.

Having realized the above deficiencies of conventional spectral Doppler ultrasound imaging devices, it is proposed herein to adapt such device such that as soon as the device detects a change of a sampled region for example due to an actuation of the trackball, angle knob or sample gate in the image-live, it automatically switches to the image-live measuring mode, preferably at full frame rate and in real-time and at full resolution. The device may then switch back to the spectrum-live measurement mode, also preferably at full frame rate and in real-time and full resolution, after a predetermined time period or after the device has detected that the sampled region has not been changed and is therefore stable for a predetermined period of time.

In other words, the proposed ultrasound imaging system may automatically switch to the image-live measurement mode preferably exactly at those instances when a user may need the color or grey-scale 2D image-live, namely then when the user wants to change a sampled region corresponding to a region of interest. Accordingly, the user may actively indicate a desired change of the sampled region for example by indicating such change on a trackball or angle knob or by moving the ultrasound transducer of the imaging device and the imaging device will detect such indicated change and automatically switch to the image-live measurement mode such that the user may see and, if necessary, correct the position or angle of the sampled region within the 2D ultrasound image. Thereby, tedious efforts for repeatedly toggling between image-live and spectrum-live measurement modes may be prevented simplifying the use of the ultrasound imaging device. Furthermore, the imaging device may be in the spectrum-live measurement mode most of the time allowing high quality spectrum analysis and is only interrupted by switching into the image-live measurement mode for the case, that a change of a sampled region is detected.

According to an embodiment of the present invention, the method further comprises automatically switching to the spectrum-live Doppler measurement mode a predetermined duration after the detection of the change of the sampled region. For example, such predetermined duration may be one second. In other words, upon detection of a change of the sampled region, the device automatically switches from the spectrum-live measurement mode to the image-live measurement mode and then, after e.g. one second, back to the spectrum-live measurement mode. Accordingly, the imaging device is within the spectrum-live measurement mode most of the time and is only interrupted for a short period of time by the image-live measurement mode. This may improve the quality of the spectrum-live measurements.

According to a further embodiment of the present invention, the method further comprises automatically switching to the spectrum-live Doppler measurement mode upon detection of the sampled region being positionally stable for a predetermined duration. In other words, the imaging device automatically switches to the image-live measurement mode upon detection of a change of the sampled region and then stays within the image-live measurement mode as long as the sampled region is still changed. The device waits until the sampled region is not changed anymore and is positionally stable for a predetermined duration such as for example one second. Only then when apparently an operator has changed the sampled region to correspond with a new desired region of interest, the device switches back to the spectrum-live Doppler measurement mode.

According to a further embodiment of the present invention, the Doppler ultrasound imaging device is controlled to operate at least in one of the spectrum-live Doppler measurement mode and the image-live measurement mode in a full frame rate mode. Preferably, the device operates in the full frame rate mode in both, spectrum-live and image-live measurement modes. Therein, the term “full frame rate mode” may indicate that images or spectra are acquired continuously and not only as a single frame update for each change of a sampled region. Furthermore, the imaging device preferably operates in a real-time mode in both, the spectrum-live and the image-live measurement modes. Therein, the term “real-time” may indicate that there is only a negligible delay in time between a real motion in the sampled region and its acquisition and display by the imaging device. Accordingly, in both measurement modes, the device may operate with the highest possible imaging quality and compromises are avoided.

According to a further embodiment of the present invention, the change of the sampled region is detected based on an actuation of a sampled region control device adapted for controlling at least one of a position, an angle, a shape, a size of the sampled region and a flow direction cursor angle. Such sampled region control device may be for example a trackball, an angle knob or a sample gate controller which may be connected to or provided on the ultrasound imaging device. In other words, the imaging device may detect that a sampled region has to be changed due to the fact that an operator actuates for example a trackball, angle knob or sample gate controller and may then automatically switch to the image-live measurement mode.

According to a further embodiment of the present invention, the Doppler ultrasound imaging device is controlled to stay in the image-live measurement mode upon manual selection of such mode by a user until manual selection of the spectrum-live measurement mode by the user. In other words, the Doppler ultrasound imaging device may have for example a switch or stylus which may be actuated by an operator. Thereby, the operator may control the imaging device to switch to the image-live measurement mode and to stay there until further action of the operator. The operator may then use the same switch or stylus or, alternatively, e.g. an additional switch or stylus to indicate that the device can return to the spectrum-live Doppler measurement mode. Such option of manual selection enables an operator to switch the imaging device into the image-live measurement mode even though a sampled region is not actually changed.

It has to be noted that aspects and embodiments of the present invention have been described mainly with reference to the proposed method for controlling a spectral Doppler ultrasound imaging device. A person skilled in the art will realize that corresponding features and characteristics may be realized for a spectral Doppler ultrasound imaging device according to the above second aspect of the present invention as well as for the computer program product and the computer-readable medium of the above third and fourth aspect of the present invention. A person skilled in the art will gather from the above and the following description that, unless other notified, in addition to any combination of features belonging to one type of subject-matter also any combination between features relating to different subject-matters, in particular between features of apparatus-type claims and features of method-type claims, is considered to be disclosed with this application.

### BRIEF DESCRIPTION OF THE DRAWINGS

Features and advantages of the present invention will be further described with respect to specific embodiments as shown in the accompanying figures but to which the invention shall not be limited.

FIG. 1 schematically shows a spectral Doppler ultrasound imaging device according to an embodiment of the present invention.

FIG. 2 shows a flow-chart for describing a method of controlling a spectral Doppler ultrasound imaging device according to an embodiment of the present invention.

The drawings are only schematical and not to scale.

### DETAILED DESCRIPTION OF THE INVENTION

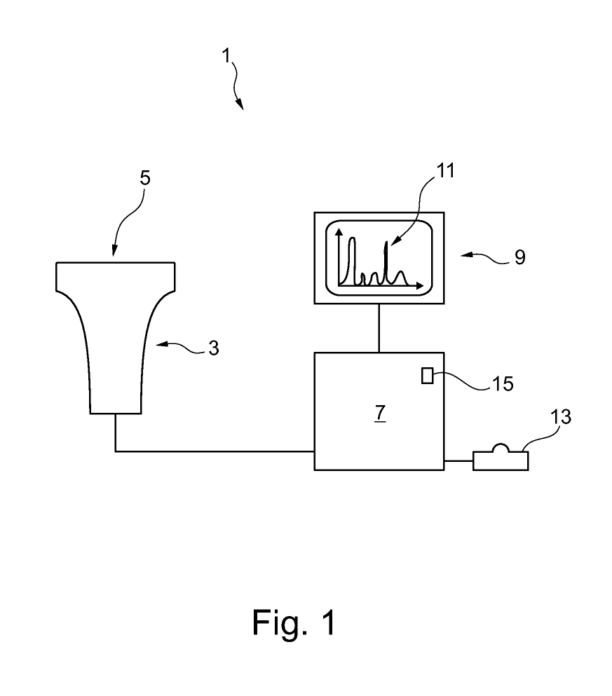

In FIG. 1, a spectral Doppler ultrasound imaging device 1 according to an embodiment of the present invention is schematically depicted. An ultrasound transducer 3 comprises an ultrasound transceiver face 5 from which ultrasound waves can be emitted into a patient's body and which may then detect reflected echoes. The transducer 3 is connected to a control device 7. The control device 7 may receive the detected echo signals from the transducer 3 and, based thereon, may display corresponding ultrasound images on a display 9. In a first operation mode, the control device 7 may control the transducer 3 to operate in an image-live measurement mode to provide color or grey-scale two-dimensional (2D) or three-dimensional (3D) ultrasound images to be displayed on the display 9. In a second mode, the control device 7 may control the transducer 3 to operate in a spectrum-live Doppler measurement mode and e.g. a quantitative analysis of velocities of motions within a sampled region may be derived from such measurements. This quantitative analysis may be displayed on the display 9 for example in the form of a graph 11 indicating velocities dependent on the time. Therein, the Doppler spectrum-live display is generally a continuously updated strip, where the horizontal x-axis represents time and the vertical y-axis represents velocity or Doppler frequency, and grey level may be related to an echo power. It may be overlaid with line graphs of peak and mean velocity.

In case, a user wants to change the sampled region, this may be indicated to the control device 7 by actuating a trackball 13. Upon such actuation, the control device 7 may switch the imaging device 1 into the image-live measurement mode such that the position and angle of the image provided by the transducer 3 may be correctly chosen by the operator. Alternatively, an operator may actuate a switch 15 to force the imaging device 1 to switch to the image-live measurement mode.

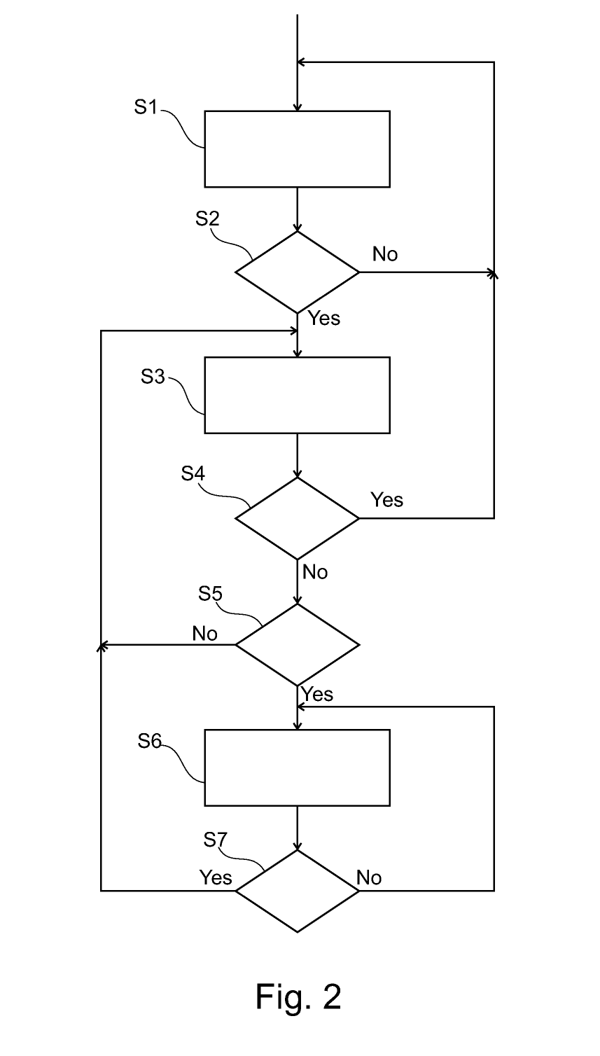

The flow-chart of FIG. 2 describes a sequence of steps S1 to S7 of a method for controlling a spectral Doppler ultrasound imaging device according to an embodiment of the present invention.

After having started the imaging device, in a first step S1, it is in an image-live measurement mode.

In step S2, it is determined whether an operator manually selects to switch to the spectrum-live measurement mode, for example, by actuating a respective switch 15. If so, the imaging device is set to the spectrum-live measurement mode, step S3. If not, the device remains in the image-live mode, step S1.

When the device has been switched into the spectrum-live mode, step S3, it is determined in step S4 whether an operator manually selects to switch to the image-live measurement mode, for example, by actuating the respective switch 15. If so, the imaging device is set to the image-live measurement mode, step S1.

If not, it is determined in step S5, whether a sampled region has been changed or not, which may be indicated by a user's actuation of the trackball 13. If so, the imaging device is set to the image-live measurement mode, step S6. If not, the imaging device remains in the spectrum-live measurement mode, step S3.

After having been set to the image-live measurement mode, step S6, it is determined in step S7 whether the sampled region has been positionally stable for a predetermined time, i.e., whether a user has finished to change a position or angle or shape of the sampled region. If so, the imaging device is switched back to the spectrum-live measurement mode, step S3. If not, the imaging device remains in the image-live measurement mode, step S6.

Features of the invention may be summarized as follows. A method for controlling a spectral Doppler ultrasound imaging device adapted for operating both in a spectrum-live Doppler measurement mode S3 and in an image-live measurement mode S1, S6 is proposed. In accordance with the present invention, the method comprises automatically switching to the image-live measurement mode S6 upon detection S5 of a change of a sampled region. A change of a sampled region may be indicated by an operator by actuating a sampled region control device 13 such as a trackball, angle knob or sample gate controller and, upon such actuation, the spectral Doppler ultrasound imaging device may automatically switch to the image-live measurement mode S6 to allow for adapting or correcting of a position and/or orientation of the sampled region. For example, after the sampled region has been positionally stable for a predetermined duration, the spectral Doppler ultrasound imaging device may be switched back S7 to the spectrum-live measurement mode S3. Thereby, unnecessary manual toggling between different measurement modes may be avoided, and quantitative spectrum-live Doppler measurement S3 is enabled being interrupted only when necessary, i.e., when a sampled region indicated inter alia by a sampled region position, a beam steer angle, a flow direction cursor angle or a size of the sampled region is changed.

It should be noted that the above-mentioned embodiments illustrate rather than limit the invention, and that those skilled in the art will be able to design many alternative embodiments without departing from the scope of the appended claims. The term “comprising” does not exclude other elements or steps, and the term “a” or “an” does not exclude a plurality of elements. It should also be noted that reference signs in the claims should not be construed as limiting the scope of the claims. The invention may be implemented by means of hardware comprising several distinct elements, and/or by means of a suitably programmed processor. In the device claim enumerating several means, several of these means may be embodied by one and the same item of hardware. The mere fact that certain measures are recited in mutually different dependent claims does not indicate that a combination of these measures cannot be used to advantage.

### List of Reference Signs

1 Doppler ultrasound imaging device

3 Ultrasound transducer

5 Ultrasound transceiver face

7 Control device

9 Display

11 Graph

13 Trackball

15 Switch

S1 Imaging device is in an image-live measurement mode

S2 Determine whether an operator manually selects to switch to the spectrum-live measurement mode

S3 Imaging device is in the spectrum-live measurement mode

S4 Determine whether an operator manually selects to switch to the image-live measurement mode

S5 Determine whether a sampled region has been changed

S6 Imaging device is in the image-live measurement mode

S7 Determine whether the sampled region has been positionally stable for a predetermined time

1. A method for controlling a spectral Doppler ultrasound imaging device (1) adapted for operating both in a spectrum-live Doppler measurement mode (S3) and in an image-live measurement mode (S1, S6), the method comprising:
automatically switching (S5) to the image-live measurement mode (S6) upon detection of a change of a sampled region.

2. The method of claim 1, further comprising automatically switching (S7) to the spectrum-live Doppler measurement mode (S3) a predetermined duration after the detection of the change of the sampled region.

3. The method of claim 1 or 2, further comprising automatically switching (S7) to the spectrum-live Doppler measurement (S3) mode upon detection of the sampled region being positionally stable for a predetermined duration.

4. The method of one of claims 1 to 3, wherein the spectral Doppler ultrasound imaging device (1) is controlled to operate at least in one of the spectrum-live Doppler measurement mode (S3) and the image-live measurement mode (S1, S6) in a full frame rate mode.

5. The method of one of claims 1 to 4, wherein the spectral Doppler ultrasound imaging device (1) is controlled to operate in both the spectrum-live Doppler measurement mode (S3) and the image-live measurement mode (S1, S6) in a real time mode.

6. The method of one of claims 1 to 5, wherein the change of the sampled region is detected (S5) based on an actuation of a sampled region control device (13) for controlling at least one of a position, an angle, a shape and a size of the sampled region and a flow direction cursor angle.

7. The method of claim 6, wherein the sampled region control device (13) comprises at least one of a trackball, an angle knob and a sample gate controller.

8. The method of one of claims 1 to 7, wherein the spectral Doppler ultrasound imaging device (1) is controlled to stay in the image-live measurement mode (S1) upon manual selection (S4) of such mode by a user until manual selection (S2) of the spectrum-live Doppler measurement mode (S3) by the user.

9. A spectral Doppler ultrasound imaging device (1) adapted for operating both in an spectrum-live Doppler measurement mode (S3) and in an image-live measurement mode (S1, (S6), the spectral Doppler ultrasound imaging device (1) comprising a control device (7) for automatically switching (S5) to the image-live measurement mode (S6) upon detection of a change of a sampled region.

10. A computer program product comprising a program code which, when executed on a computer, is adapted to control a spectral Doppler ultrasound imaging device (1) according to the method of one of claims 1 to 8.

11. A computer readable medium having a computer program product according to claim 10 stored thereon.
